(19) United States
(12) Patent Application Publication
(10) Pub. No.: US 2026/0102263 A1
(43) Pub. Date: Apr. 16, 2026

(54) PROSTHETIC JOINT ACTUATION SYSTEM

(71) Applicant: Össur Iceland ehf, Reykjavik (IS)

(72) Inventors: David Landry, St-Jean-Chrysostôme, QC (CA); Guðni Ingimarsson, Reykjavik (IS); François Heremans, Les Plans-sur-Bex (CH)

(21) Appl. No.: 19/358,956

(22) Filed: Oct. 15, 2025

Related U.S. Application Data
(60) Provisional application No. 63/707,595, filed on Oct. 15, 2024.

(51) Int. Cl. A61F 2/70; A61F 2/60

(52) U.S. Cl. CPC A61F 2/70; A61F 2/60; A61F 2002/607; A61F 2002/701

(57) ABSTRACT

An actuation assembly for a prosthetic device may include an output arm, a motor, and a drive unit. The drive unit may include an input coupled to the motor, an output coupled to the output arm, and a drive transmission. The drive transmission may be configured to transmit rotation of the input to the output. The motor and the drive unit may be axially aligned.

### INCORPORATION BY REFERENCE TO ANY PRIORITY APPLICATIONS

This application claims priority to U.S. Provisional Application No. 63/707,595, filed Oct. 15, 2024, which is incorporated herein by reference. Any and all applications for which a foreign or domestic priority claim is identified in the Application Data Sheet as filed with the present application are hereby incorporated by reference under 37 CFR 1.57.

### BACKGROUND

### Field

The present disclosure relates to a prosthetic joint, and more particularly, aspects of the present disclosure relate to actuation systems for a powered prosthetic joint.

### Description of the Related Art

A few types of joint actuation systems for prosthetic devices are known in the art. Usually, joint actuation systems form part of a prosthetic device and include a housing for a motor. In order to generate the speed and torque required to properly support movement (e.g., locomotion, ambulation), the joint actuation systems typically include a transmission assembly between the motor and an output arm of the joint actuation system. The transmission typically includes a strain wave gear assembly. However, strain wave gear assemblies generate undesirable vibration and noise during operation.

### SUMMARY

For purposes of this summary, certain aspects, advantages, and novel features are described herein. It is to be understood that not necessarily all such advantages may be achieved in accordance with any particular aspect. Thus, for example, those skilled in the art will recognize the disclosures herein may be carried out in a manner that achieves one or more advantages taught herein without necessarily achieving other advantages as may be taught or suggested herein.

In some aspects, an actuation assembly for a joint mechanism of a prosthetic device may include an output arm; a motor; and a drive unit including an input coupled to the motor, an output coupled to the output arm, and a drive transmission, wherein the drive transmission may be configured to transmit rotation of the input to the output, wherein the motor and the drive unit may be axially aligned, and wherein the drive transmission may include a traction transmission or a friction transmission.

In some aspects, the drive transmission may include a planetary drive transmission.

In some aspects, the drive transmission may include a compound-planetary drive transmission.

In some aspects, the drive transmission may include a plurality of stages.

In some aspects, each stage may include a planetary drive configuration.

In some aspects, a reduction ratio of the drive transmission may be between 35:1 and 80:1.

In some aspects, a reduction ratio of the drive transmission may be between 25:1 and 60:1.

In some aspects, the motor may include a brushless motor.

In some aspects, the motor may include an axial flux motor.

In some aspects, the motor may include a radial flux motor.

In some aspects, windings of the motor may include a printed circuit board.

In some aspects, the printed circuit board may include a flexible printed circuit board.

In some aspects, windings of the motor may include a printed circuit board.

In some aspects, the prosthetic device may include a lower limb prosthetic device.

In some aspects, the output arm may include a compliant transmission.

In some aspects, the compliant transmission may include two links, wherein a first link of the two links is compliant.

In some aspects, the compliant transmission may include a single flexible output arm.

In some aspects, the output arm may be rigid.

In some aspects, a joint mechanism for a prosthetic device may include a housing including a prosthetic connector; and an actuation assembly positioned in the housing, the actuation assembly may include an output arm; a motor; and a drive unit including an input coupled to the motor, an output coupled to the output arm, and a drive transmission, the drive transmission may be configured to transmit rotation of the input to the output, a rotational axis of the motor and a rotation axis of the drive unit may be parallel, and the drive transmission may include a traction transmission or a friction transmission.

In some aspects, the drive unit may include a measurement shaft, and an encoder configured to measure a rotation position of the measurement shaft.

In some aspect, the measurement shaft may extend axially through the input.

In some aspects, the housing may include a drive unit housing coupled to a motor housing, wherein the drive unit may be positioned in the drive unit housing and the motor may be positioned in the motor housing.

In some aspects, the prosthetic connector may be coupled to the motor housing.

In some aspects, the prosthetic device may include a lower limb prosthetic device.

In some aspects, the planetary drive transmission may include a plurality of stages.

In some aspects, the planetary drive transmission may include a plurality of stages, and each stage may include a planetary gear.

In some aspects, the planetary drive transmission may include an input shaft; a plurality of planet rollers; an outer ring positioned circumferentially around the input shaft; a plurality of output rollers; and an output shaft, wherein the plurality of output rollers may be coupled to the output shaft, wherein the plurality of planet rollers may be positioned between the input shaft and the outer ring, and the plurality of output roller may be positioned between the plurality of planet rollers, wherein the plurality of planet rollers may be configured to circumferentially rotate around the input shaft when the input shaft rotates and the plurality of planet rollers may be configured to rotate the output rollers.

In some aspects, the drive transmission may include a compound-planetary drive transmission.

In some aspects, the compound planetary drive transmission may include a first sun roller; a second sun roller positioned coaxially around the first sun roller; a ring annulus including an outer ring annulus and an inner ring annulus; and a plurality of planet rollers positioned between the first sun roller and the ring annulus, so outer portions of each planet roller contacts the first sun roller and the outer ring annulus, and an inner portion of each planet roller contact the inner ring annulus and the second sun roller.

In some aspects, the input of the drive unit may include the first sun roller and the output of the drive unit may include the outer ring annulus, wherein the outer ring annulus may be coupled to the motor housing and the output arm, wherein the inner ring annulus may be coupled to the drive unit housing, and wherein the prosthetic connector may be coupled to the drive unit housing.

In some aspects, when the first sun roller rotates the first sun roller may be configured to rotate the plurality of planet rollers, and wherein the plurality of planet rollers may be configured to rotate the inner annulus relative to the outer ring annulus to rotate the prosthetic connector relative to the output arm.

In some aspects, the output arm and the motor housing may be coupled to a shank portion of the prosthetic device.

In some aspects, a diameter of the inner portion of each planet roller may be larger than a diameter of the outer portions of each planet roller.

In some aspects, a sensor assembly may be configured to determine an angle between a limb segment of a user and a shank portion of the prosthetic device, the sensor assembly may include a magnet axially aligned with actuation assembly and a sensor configured to measure rotation of the magnet.

In some aspects, an actuation assembly for a joint mechanism of a prosthetic device may include an output arm, a motor; and a drive unit including an input coupled to the motor, an output coupled to the output arm, and a drive transmission, the drive transmission may be configured to transmit rotation of the input to the output, and the drive transmission may include a cycloid drive transmission.

In some aspects, the drive transmission may include a gear eccentrically coupled to the input such that when the input rotates about a transverse axis the gear rotates in a cycloidal path about the input, a plurality of pins positioned circumferentially around the gear, the gear a plurality of teeth, the plurality of teeth of the gear may be configured to interact with the plurality of pins to generate torque, and the drive transmission may further include a transformation member coupled to the gear and the output, and configured to convert rotation of the gear in the cycloidal path to rotation about the transverse axis in order to rotate the output about the transverse axis.

In some aspects, the drive transmission may include a gear eccentrically coupled to the input.

In some aspects, rotation of the input may cause a gear of the drive transmission to rotate in a cycloidal path about the input.

In some aspects, the drive transmission may include a first stage and a second stage, a first gear of the first stage may be eccentrically coupled to the input and a second gear of the second stage may be eccentrically coupled to the input, and the first gear and the second gear may be offset from the first gear by 180 degrees.

In some aspects, an actuation assembly for a joint mechanism of a prosthetic device may include an output arm, a motor; and a drive unit including an input coupled to the motor, an output coupled to the output arm, and a drive transmission, the drive transmission may be configured to transmit rotation of the input to the output, and the drive transmission may be eccentrically coupled to the input.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1A illustrates a perspective view of an example of a prosthetic device.

FIG. 1B illustrates a perspective view of the example prosthetic device of FIG. 1A with a cover of the prosthetic device removed showing partially a joint mechanism.

FIG. 2A illustrates a cross-sectional view of a joint mechanism of a prosthetic device.

FIG. 2B illustrates a cross-sectional view of a drive unit and motor of a joint mechanism of a prosthetic device.

FIG. 3 illustrates an example of a stage of a drive unit.

FIG. 4A illustrates a perspective view of an example prosthetic device of with a cover of the prosthetic device removed showing partially a joint mechanism with an axial flux motor.

FIG. 4B illustrates an exploded view of the axial flux motor of the example prosthetic device of FIG. 4A.

FIG. 4C illustrates a cross-sectional view of the joint mechanism of the example prosthetic device of FIG. 4A.

FIG. 5 illustrates a cross-section view of another joint mechanism of an example prosthetic device.

FIG. 6A illustrates an exploded view of another drive unit of a joint mechanism of a prosthetic device.

FIG. 6B illustrates a partial cross-sectional view of the drive unit of FIG. 6A.

FIG. 7A illustrates a cross-sectional view of another joint mechanism of a prosthetic device with the joint mechanism having radial flux motor.

FIG. 7B illustrates a cross-sectional view of another joint mechanism of a prosthetic device with the joint mechanism having an axial flux motor.

FIG. 8A illustrates a cross-sectional view of another joint mechanism of a prosthetic device with the joint mechanism having a motor with PCB windings, and an example strain wave drive transmission.

FIG. 8B illustrates a cross-sectional view of another joint mechanism of a prosthetic device with the joint mechanism having a motor with PCB windings and an example traction drive transmission.

### DETAILED DESCRIPTION

Although several aspects, examples, and illustrations are disclosed below, it will be understood by those of ordinary skill in the art that the system, methods, and devices described herein extend beyond the specifically disclosed aspects, examples, and illustrations and includes other uses of the system, methods, and devices and obvious modifications and equivalents thereof. The terminology used in the description presented herein is not intended to be interpreted in any limited or restrictive manner simply because it is being used in conjunction with a detailed description of certain specific aspects of the disclosure. In addition, aspects of the disclosure can include several novel features and no single feature is solely responsible for its desirable attributes or is essential to practicing the system, methods, and devices herein described.

Strain wave gears have a high torque density, which can allow prosthetic joints to be small and compact, while providing proper power output levels at acceptable efficiency levels. The torque capacity of a strain wave gear is based on the diameter and gear ratio of the strain wave gear, and the gear ratio and torque transfer capacity are coupled in strain wave gears, which can limit the effectiveness of strain wave gears when dealing with small diameters, and high torque and low gear ratio requirements. Additionally, the diameter and gear ratio of a strain wave gear in a prosthetic joint is limited by the space available in a housing of the prosthetic joint. In prosthetic joints, a high torque density at a low gear ratio can improve transparency of the joint mechanism. Transparency is a measure of how seamless the motor motion is perceived by a user (e.g., an amputee). Transparency of a prosthetic joint correlates with back-driveability and reflected inertia at an output arm of the prosthetic joint. Back-driveability and reflected inertia are related to the gear ratio of the joint mechanism. As the gear ratio increases, the reflected inertia of the motor rotor increases, making it difficult for the motor and/or the output arm to rotate fast and rapidly change speeds. In order to reduce or minimize a discrepancy between the movement a user expects and the movement output by the joint mechanism, a high gear ratio system requires finer motion controls than a low gear ratio system. Additionally, higher gear ratios require higher motor speeds to achieve the required output speed for human locomotion tasks.

A more transparent system can allow a user to more easily back-drive the system so the user can control movement of the motor and/or the prosthetic joint more accurately. Back-driveability can be achieved actively with sensors and a controller, or passively by having a joint mechanism with a low inertia, low friction, and a low gear ratio. A joint mechanism with a low inertia, low friction, and a low gear ratio can be implemented without accurate knowledge of the environment and interaction between the user and the prosthetic device.

Drive systems with strain wave gears typically generate vibration as the gears rotate. The vibration is caused by the meshing and unloading of the teeth as the flex-spline and circular spline interact. The vibration generated by strain wave gears, when rotating at speeds required to achieve the required output speed for human locomotion tasks, results in noisy prosthetic joint and generates low-frequency vibration that can excite the natural frequency of structural components of a prosthetic device, resulting in additional noise and vibration generation during operation.

In order to minimize the stigma associated with a noisy prosthetic device and the associated functional loss, it can be desirable to reduce or minimize the noise and vibration generated by the prosthetic joint mechanism without affecting ability of the motor to generate the speed and torque required to properly support human locomotion (e.g., ambulation) tasks. The noise generated by the joint mechanism can be reduced or minimized by reducing or minimizing the speed of the motor, which can reduce the noise and vibration generated from the rotating components of the motor, such as the rotor, bearings, and shafts. For typical human locomotion tasks, minimizing the motor speed requires a transmission with relatively low reduction ratios.

In a lower limb prosthetic, the motor operates under oscillatory movement conditions, and the lower limb prosthetic periodically transfers between a stance phase and a swing phase of a gait cycle. During the stance phase, and/or during the transfer between the stance phase and the swing phase, the prosthetic can be subject to large loading forces. Presence of backlash or play in a joint mechanism that uses teeth to transfer motion between components (e.g., gears) can cause discontinuous movement, resistance, or loss of support in the joint mechanism, particularly when the prosthetic is subject to large loading forces.

The prosthetic devices disclosed herein can address one or more of the disadvantages of existing joint actuation mechanisms discussed above, and/or other problems of the current prosthetic devices, and include one or more of the advantages disclosed herein, and/or other advantages. In order to reduce or minimize the noise and vibration generated from meshing and unloading of teeth in a typical gear transmission, the prosthetic devices herein can instead include a traction or friction transmission. Traction or friction transmission can provide the torque density required for a joint mechanism in a lower limb prosthetic to control movement of the lower limb prosthetic during human locomotion tasks. A traction transmission has smooth, unequal size wheels in driving contact and a lubricant film that transmits power between the wheels. A friction transmission also has smooth, unequal size wheels in driving contact, but no lubricant film is used, and dry friction transmits power between the wheels.

The reduction ratio, diameter, and torque capacity of a traction or friction transmission are uncoupled. The traction or friction transmission can be more easily modified to produce a desired reduction ratio and/or torque capacity while being sized to fit in a prosthetic device. Additionally, when compared to a strain wave gear, the traction or friction transmission can produce a lower the reduction ratio and/or a higher torque capacity with a same diameter. For example, traction or friction transmission can run a torque level of 80 Nm at a reduction ratio of about 42:1, with a diameter of 62 mm, whereas a strain wave gear with a comparable gear ratio and diameter can only provide a torque level of about 44 Nm. A lower reduction ratio can allow the motor to run at a lower speed, which can reduce vibration generated by unbalanced rotating components in the motor. Furthermore, lower reduction ratio can reduce the reflected inertia of the motor, which can increase back-drivability of the actuation assembly and/or the prosthetic device.

Since traction and friction transmissions do not include teeth that transfer power between components of the transmission, contact between input and output of the transmission is continuous. Therefore, traction and friction transmissions have a higher stiffness when compared to a strain wave gear. Additionally, there is no backlash between the wheels of the transmission. Furthermore, traction and friction transmissions are overload safe, since an overload would only cause slipping between the input and output of the transmission Additionally, traction and friction transmissions generate less noise and vibration when compared to a gear transmission. Noise is only generated in traction and friction transmissions from the rolling contact between components, which is significantly quieter than the noise generated by the meshing and unloading of teeth in typical gear transmissions. Since there are no teeth in traction and friction transmissions, there is no vibration generated by the meshing and unloading of teeth. Accordingly, traction and friction transmission can reduce or minimize excitation of the natural frequency of structural components of a prosthetic device, which can generate additional noise.

Additionally, the prosthetic devices herein can include an actuation assembly with an axially aligned transmission (e.g., drive unit, speed reducer) where a rotation axis of the motor and drive transmission are colinear. An axially aligned transmission can maximize the torque output by a motor sized to fit in the volume of a housing of the prosthetic joint. The axially aligned transmission can be arranged to achieve a desired reduction ratio. For example, axially aligned transmission can be configured as a planetary reducer, a compound-planetary reducer, a cycloidal reducer, or a Wolfrom reducer.

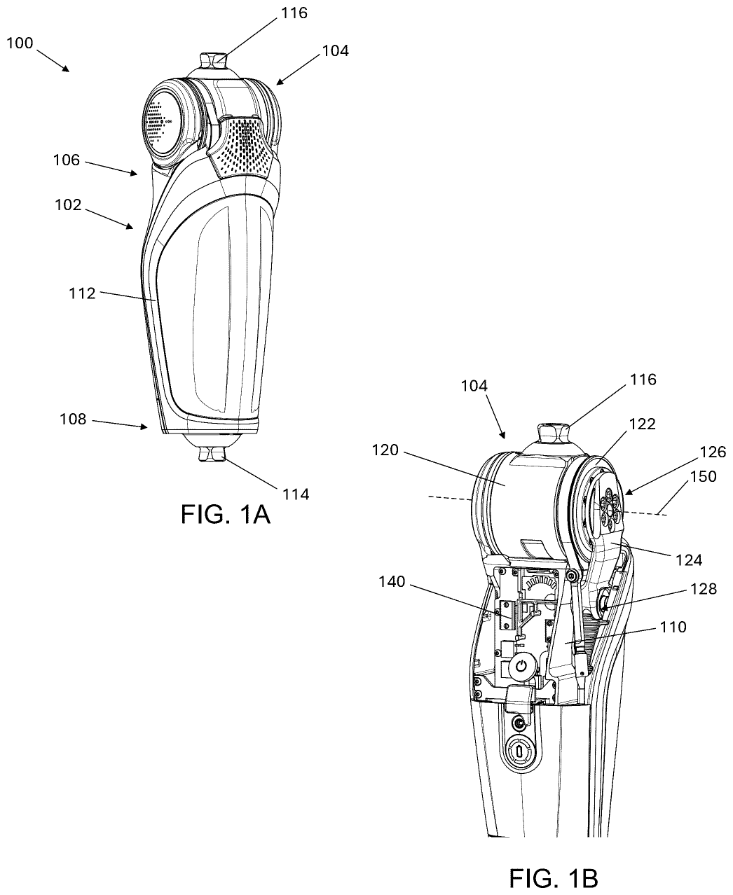

FIGS. 1A and 1B illustrate a prosthetic device 100. The prosthetic device may include a shank portion 102 and a joint mechanism 104. The prosthetic device 100 may include a lower limb prosthetic. The prosthetic device 100 may include a prosthetic knee. Accordingly, the shank portion 102 may include a lower leg and the joint mechanism 104 may include a knee joint.

The shank portion 102 may extend between a proximal end 106 and a distal end 108. The shank portion 102 may include a shank structure 110 (shown in FIG. 1B), such as a frame, and a cover 112 (shown in FIG. 1A) positioned over the shank structure 110. The shank portion 102 may include or couple to a distal connector 114 at the distal end 108 of the shank portion 102. In one example, the distal connector 114 can be a pyramid connector. The distal connector 114 may couple the prosthetic device 100 to a prosthetic ankle and/or a prosthetic foot. The joint mechanism 104 may be positioned at the proximal end 106 of the shank portion 102. The joint mechanism 104 may include a proximal connector 116. In one example, the proximal connector 116 can be a pyramid connector. The proximal connector 116 may be connected to another adjacent prosthetic portion, such as a common socket, (not shown) that is mountable to a limb segment (e.g., leg stump) of a user. The joint mechanism 104 may be positioned between the shank portion 102 and the prosthetic portion and/or the limb (e.g., leg stump) of the user. The joint mechanism 104 may rotate the shank portion 102 relative to the prosthetic portion and/or the limb (e.g., leg stump) of the user.

As shown in FIG. 1B, the joint mechanism 104 may include a housing 120, a drive unit 122, and/or an output arm 124. The proximal connector 116 may be coupled to the housing 120. The drive unit 122 may be coupled to the output arm 124. The drive unit 122 may rotate the output arm 124 about a transverse axis 150 extending through the drive unit 122. The drive unit 122 may be coupled to a proximal end 126 of the output arm 124. A distal end 128 of the output arm 124 may be coupled to the shank structure 110. When the drive unit 122 rotates the output arm 124 about the transverse axis 150, the drive unit 122 may (simultaneously) rotate the shank structure 110 about the transverse axis 150. Accordingly, the output arm 124 may transmit rotation of the drive unit 122 to the shank structure 110.

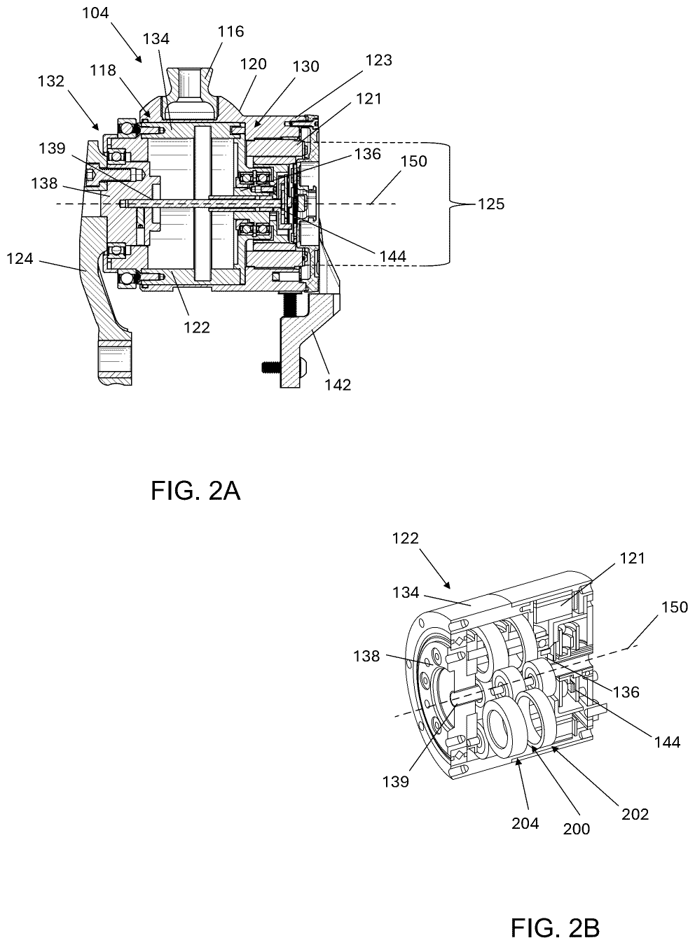

The prosthetic device 100 may include a controller 140. The controller 140 may be electrically connected to the joint mechanism 104. The controller 140 may control a motor 121 (shown in FIGS. 2A and 2B). The controller 140 may control the motor 121 based on signals received from one or more sensors (e.g., force sensors, inertial measurement units (IMUs), gyroscopes, accelerometers, etc.) mounted on or in communication with the prosthetic device 100. In one example, the motor 121 is an electric motor (e.g., whose operation is controlled by the controller 140).

FIGS. 2A and 2B illustrate a cross-sectional view of the joint mechanism 104. The joint mechanism 104 may include an actuation assembly 118. The actuation assembly 118 may be positioned in the housing 120. The actuation assembly 118 may include the motor 121, the drive unit 122, and/or the output arm 124. The motor 121 may be positioned in (e.g., rigidly coupled to) a motor housing 123 of the housing 120. The motor 121 may be coupled to the drive unit 122 on a first side 130 of the drive unit 122 such that a rotational axis (e.g., the transverse axis 150) of the motor 121 and a rotational axis (e.g., the transverse axis 150) of the drive unit 122 are parallel. The output arm 124 may be coupled to a second side 132 of the drive unit 122 opposite the first side 130 of the drive unit 122, either directly or via a compliant member. The actuation assembly 118 may include an axially aligned actuation assembly and/or the motor 121, the drive unit 122, and the output arm 124 may be axially aligned along the transverse axis 150. In some embodiments, the motor 121 and/or the drive unit 122 may be positioned in the shank structure 110.

The drive unit 122 may include an input shaft 136, and/or an output shaft 138. The drive unit 122 may be positioned in (e.g., rigidly coupled to) a drive unit housing 134 of the housing 120. The drive unit housing 134 may be coupled to the motor housing 123 to form the housing 120. In one example, the drive unit housing 134 and the motor housing 123 form the housing as a single piece (e.g., a monolithic, seamless piece). The housing 120 may be coupled to the shank structure 110 (shown in FIG. 1B) with a support block 142. The support block 142 may be rotationally coupled to the housing 120 and/or the drive unit housing 134. The support block 142 may couple the housing 120 to the shank structure 110 of the shank portion 102 without restricting the rotational movement of the shank structure 110 relative to the output shaft 138 of the drive unit 122 and/or the motor 121. The support block 142 may prevent or inhibit the joint mechanism 104 from rotating relative to the shank structure 110 about a longitudinal axis through the shank portion 102.

The input shaft 136 of the drive unit 122 may be coupled to the motor 121. The motor 121 may rotate the input shaft 136 about the transverse axis 150. The input shaft 136 may be coupled to the output shaft 138. As shown in FIG. 2B, the drive unit 122 may include a drive transmission 200 positioned between the input shaft 136 and the output shaft 138. The drive transmission 200 may be positioned in the drive unit housing 134. The drive transmission 200 may couple the input shaft 136 to the output shaft 138. The drive transmission 200 may transfer rotation of the input shaft 136 to the output shaft 138 so the output shaft 138 rotates about the transverse axis 150. The drive transmission 200 may include a reduction (e.g., gear) ratio. The reduction ratio of the drive transmission 200 may include a reduction ratio of 2:1, 5:1, 10:1, 15:1, 20:1, 25:1, 30:1, 35:1, 40:1, 42:1, 45:1, 50:1, 55:1, 60:1, 65:1, 70:1, 80:1, 90:1, 100:1, and/or any value between the aforementioned values. In some implementations, the reduction ratio of the drive transmission 200 may include a reduction ratio between 10:1 and 100:1. In some implementations, the reduction ratio of the drive transmission 200 may include a reduction ratio between 35:1 and 80:1. The drive transmission 200 may be an axially aligned speed reducer. Accordingly, the drive transmission 200 may reduce the rotation speed of the output shaft 138 relative to the input shaft 136 and/or increase a torque output by the output shaft 138. In embodiments where the drive transmission 200 includes a plurality of stages, a reduction ratio of one or more of the stages may be less than 1:1, and a reduction ratio of one or more of the stages may be greater than 1:1 such that the reduction ratio of the drive transmission 200 is greater than 1:1.

The drive transmission 200 may include a traction or friction drive unit. The drive transmission 200 may not include teeth (e.g., it excludes teeth) between the input shaft 136 and the output shaft 138 and/or contact between the input shaft 136 and the output shaft 138 may be continuous. Accordingly, backlash between the input shaft 136 and the output shaft 138 of the drive transmission 200 may be reduced or minimized or eliminated, and/or noise generated by the drive transmission 200 may be reduced or minimized.

The output shaft 138 of the drive unit 122 may be coupled to the output arm 124 at the proximal end 126 of the output arm 124. When the output shaft 138 rotates about the transverse axis 150, the output shaft 138 may cause the output arm 124 to rotate about the transverse axis 150. Accordingly, the output arm 124 may rotate the shank structure 110 (shown in FIG. 1B) when the output shaft 138 rotates the output arm 124. The output arm 124 may include a rigid output arm or a compliant transmission element. For example, the compliant transmission element may include two links and a first link of the two links may include a plurality of stacked spring elements, or the compliant transmission element may include a single flexible output arm.

The input shaft 136 of the drive unit 122 may be hollow. The drive unit 122 may include a measurement shaft 139 extending axially through the input shaft 136. The measurement shaft 139 may be coupled to the output shaft 138 and an encoder 144 positioned in the motor housing 123. The encoder 144 may include a magnetic encoder, a mechanical encoder, and/or an optical encoder. The encoder 144 may include an incremental encoder or an absolute encoder. The encoder 144 may include a single turn magnetic encoder. The measurement shaft 139 may rotate about the transverse axis 150 with the output shaft 138. The encoder 144 may measure a rotational position of the measurement shaft 139 about the transverse axis 150 to determine a rotational position of the output shaft 138 relative to the motor 121 (and therefore the housing 120). The rotational position of the output shaft 138 relative to the motor 121 may be used to determine an angle of flexion of the prosthetic (e.g., an angle between the shank structure 110 and the proximal connector 116). The controller 140 may control the motor 121 based on at least in part on the angle of flexion of the prosthetic. The encoder 144 may include a high-speed encoder (e.g., sensor). Accordingly, the encoder 144 may enable full field-oriented control of the motor commutation of the motor 121 by the controller 140. Therefore, torque ripple may be reduced or minimized, which may reduce or minimize noise generated by the motor 121.

As shown in FIG. 2B, the drive transmission 200 may include a first stage 202 and a second stage 204. The first stage 202 may be coupled to the input shaft 136 and an input of the second stage 204. The second stage 204 may be coupled to an output of the first stage 202 and the output shaft 138. The first stage 202 and the second stage 204 may each include planetary-type drive stages. In some embodiments, the first stage 202 may include a planetary-type drive stage, and the second stage 204 may include different stage configuration, or vice versa. The first stage 202 and the second stage 204 may include a reduction ratio. The reduction ratio of the first stage 202 may be a same reduction ratio as the reduction ratio of the second stage 204. The reduction ratio of the drive transmission 200 may include a product of the reduction ratio of the first stage 202 and the second stage 204.

Although the drive transmission 200 is described as including a first stage 202 and a second stage 204, the drive transmission 200 may include any number of stages. For example, the drive transmission 200 may include one (1) stage, two (2) stages, three (3) stages, four (4) stages, five (5) stage, and/or six (6) stages. The number of stages in the drive transmission 200 may be based on a desired reduction ratio of the drive transmission 200, a desired maximum torque output by the drive unit 122, a desired rotational velocity of the output arm 124, a size of the motor 121, and/or any other desired factor. In some embodiments, each stage may include a same drive stage configuration (e.g., planetary, compound-planetary, etc.). In some embodiments, one or more stage may include a different drive stage configuration.

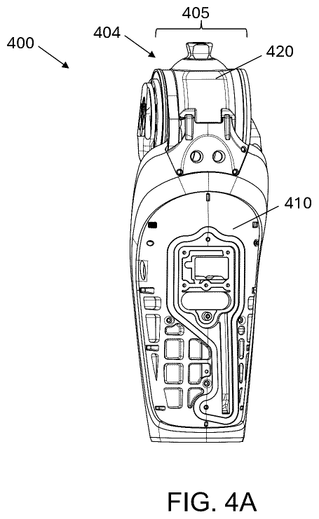

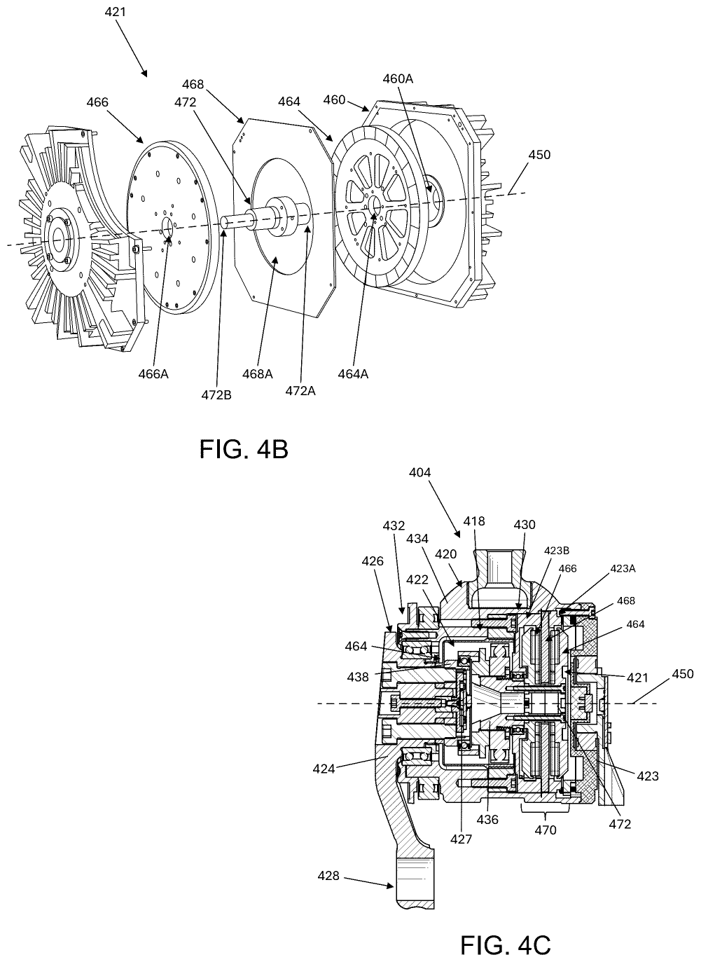

The motor 121 may include a brushless DC electric motor and/or any other type of motor used in prosthetic devices. As shown in FIG. 2A, the motor 121 may include a diameter 125. The diameter 125 of the motor 121 may be maximized in order to maximize the maximum torque the motor 121 is capable of generating. The diameter 125 may be about 10 mm, about 20 mm, about 40 mm, about 50 mm, about 60 mm, about 70 mm, about 80 mm, about 90 mm, about 100 mm, and/or any value between the aforementioned values. The reduction ratio of the drive transmission 200 may increase an output torque generated by the actuation assembly 118 (e.g., the motor 121) and/or applied to the output arm 124. For example, the drive transmission 200 may include a reduction ratio of 42:1, which may allow a motor 121 with a diameter of 50 mm to generate an output torque of 80 Nm. In some embodiments, the motor 121 may include a radial flux motor. In some embodiments, the motor 121 may include the axial flux motor 421 described with reference to FIGS. 4A-4C.

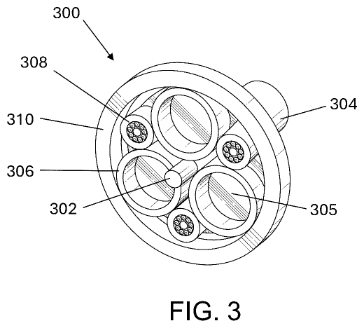

FIG. 3 illustrates an example of a stage 300 of the drive transmission 200. The first stage 202 and/or the second stage 204 may include any of the features of the stage 300. The stage 300 may include a traction or friction drive. The stage 300 may be or include a planetary gear configuration. A traction drive includes a lubricant film between components of the stage 300. The lubricant film may transmit power or movement (e.g., rotation) between the components of the stage 300. A friction drive does not include a lubricant film and relies on friction between the components of the stage 300 to transmit power or movement (e.g., rotation) between the components.

The stage 300 may include an input shaft 302, and output shaft 304, planet rollers (e.g., hollow rollers) 306, and/or output rollers (e.g., stop rollers) 308. The input shaft 302 may be coupled to a motor, such as the motor 121, or an output shaft 304 of a previous stage 300. The outer ring 310 may be positioned circumferentially around the input shaft 302. The planet rollers 306 may be positioned between the input shaft 302 and an outer ring 310. The outer ring 310 may include a housing, such as the drive unit housing 134. The planet rollers 306 may contact the input shaft 302 and the outer ring 310. The planet rollers 306 may be sized such that a pressure is applied between the input shaft 302 and the planet rollers 306, and the planet rollers 306 and the outer ring 310. Accordingly, when the input shaft 302 rotates about a transverse axis 320, the input shaft 302 may cause the planet rollers 306 to rotate.

The outer ring 310 may be stationary. Accordingly, when the input shaft 302 causes the planet rollers 306 to rotate, the planet rollers 306 may circumferentially rotate around the input shaft 302. The planet rollers 306 may be circumferentially spaced around the input shaft 302, and the output rollers 308 may be positioned between the planet rollers 306. The output rollers 308 may be coupled to a carrier 305 of the output shaft 304. The carrier 305 may hold the output rollers 308 is a fixed position relative to each other, and with respect to the planet rollers 306 so the output rollers 308 contact the planet rollers 306. Accordingly, when the planet rollers 306 rotate circumferentially around the input shaft 302 (e.g., around the transverse axis 320), the planet rollers 306 may cause the output rollers 308, the carrier 305, and/or the output shaft 304 to rotate.

The stage 300 may include a reduction (e.g., gear) ratio. The reduction ratio may be calculated as a ratio of the diameter of the outer ring 310 to the diameter of the input shaft 302 plus one. The reduction ratio of the stage 300 may include a ratio of 2:1, 3:1, 4:1, 5:1, 6:1, 7:1, 8:1, 9:1, 10:1, 11:1, 12:1, 13:1, and/or any value between the aforementioned values.

FIG. 4A illustrates a prosthetic device 400. The prosthetic device 100 can have any of the features of the prosthetic device 400, and the prosthetic device 400 can have any features of the prosthetic device 100. The prosthetic device 400 may include a joint mechanism 404 with an axial flux motor 421, as shown in FIGS. 4B and 4C. The axial flux motor 421 may reduce or minimize an axial length (e.g. width) required to mount the axial flux motor 421 in the joint mechanism 404. Accordingly, an axial length (e.g., width) 405 of the housing 420 may be reduced or minimized. Therefore, the axial length 405 of the housing 420 may be smaller than an axial length of a housing of a prosthetic device with a radial flux motor.

FIG. 4B illustrates an exploded view of the axial flux motor 421. The axial flux motor 421 may include a first case 460, a second case 462, a first rotor 464, a second rotor 466, and/or a stator 468. The first case 460 and the second case 462 may be coupled together. The first rotor 464, the second rotor 466, and/or the stator 468 may be positioned between the first case 460 and the second case 462. The stator 468 may be positioned between the first rotor 464 and the second rotor 466. The first rotor 464 and the second case 462 may be positioned on opposite sides of the stator 468. The first rotor 464 may be positioned between the stator 468 and the first case 460. The second rotor 466 may be positioned between the stator 468 and the second case 462.

The first rotor 464, the second rotor 466, and/or the stator 468 may be substantially flat. The first rotor 464, the second rotor 466, and/or the stator 468 may be positioned perpendicular to the transverse axis 450. Accordingly, an axial length (e.g., width) 470 of the axial flux motor 421 (shown in FIG. 4C), may be reduced or minimized.

In order to reduce or minimize a thickness (e.g., an axial length) of the stator 468, the stator 468 may include a printed circuit board (PCB). Therefore, the axial length 470 of the axial flux motor 421 may be reduced or minimized. Furthermore, making the stator 468 a PCB may increase the manufacturing repeatability of the stator 468 and/or the axial flux motor 421, reduce or minimized a cost of manufacturing of the stator 468 and/or the axial flux motor 421, and/or allow the axial flux motor 421 to have a high torque density. Additionally, making the stator 468 a PCB may reduce or minimize a weight of the stator 468.

The axial flux motor 421 may include a shaft 472. The shaft 472 may be positioned so the shaft 472 extends along the transverse axis 450. A first end 472A of the shaft 472 may be positioned in a recess 460A in the first case 460. The first rotor 464 may include an opening 464A extending through the first rotor 464, the second rotor 466 may include an opening 466A extending through the second rotor 466, and/or the stator 468 may include an opening 468A extending through the stator 468. The shaft 472 may extend through the openings 464A, 466A, 468A. The shaft 472 may extend through an opening 462A in the second case 462 so a second end 472B of the shaft 472 is positioned outside of the second case 462. The first rotor 464 and the second rotor 466 may be coupled to the shaft 472 such that when the stator 468 rotates the first rotor 464 and the second rotor 466, the first rotor 464 and the second rotor 466 may rotate the shaft 472.

FIG. 4C illustrates a cross-sectional view of the joint mechanism 404. The joint mechanism 404 may include a housing 420 and an actuation assembly 418 positioned in the housing 420. The actuation assembly 418 may include the axial flux motor 421, a drive unit 422, and/or an output arm 424. The axial flux motor 421 may be positioned in a motor housing 423 of the housing 420. The drive unit 422 may be positioned in a drive unit housing 434 of the housing 420. The axial flux motor 421 may be coupled to the drive unit 422 on a first side 430 of the drive unit 422. The output arm 424 may be coupled to the drive unit 422 on a second side 432 of the drive unit 422 opposite the first side 430 of the drive unit 422.

The axial flux motor 421 may rotate an input shaft 436 of the drive unit 422 about the transverse axis 450. The input shaft 436 of the drive unit 422 may include the shaft 472 of the axial flux motor 421 or the input shaft 436 may be coupled to the shaft 472 of the axial flux motor 421. The input shaft 436 may be coupled to an output shaft 438 of the drive unit 422. The drive unit 422 may include a drive transmission 427 positioned between the input shaft 436 and the output shaft 438. The drive transmission 427 may couple the input shaft 436 to the output shaft 438. The drive transmission 427 may include a traction or friction drive unit, such as drive transmission 200 or drive transmission 527, or a strain wave gear drive unit. The drive transmission 427 may transfer rotation of the input shaft 436 to the output shaft 438 so the output shaft 438 rotates about the transverse axis 450.

The output shaft 438 of the drive unit 422 may be coupled to the output arm 424 at a proximal end 426 of the output arm 424. The output shaft 438 may rotate the output arm 424 about the transverse axis 450 when the output shaft 438 rotates. A distal end 428 of the output arm 424 may be coupled to a shank structure 410 (shown in FIG. 4A) of the prosthetic device 400. Accordingly, the output shaft 438 may rotate the shank structure 410 relative to the joint mechanism 404 when the output shaft 438 rotates about the transverse axis 450.

The axial flux motor 421 may be positioned in the motor housing 423 so the shaft 472 extends towards the drive unit 422. Accordingly, the axial flux motor 421 may be positioned so the second rotor 466 is positioned between the first rotor 464 and the drive unit 422. The motor housing 423 may include a first stator holder 423A and a second stator holder 423B. The first case 460 may be integrated with the first stator holder 423A, and the second case 462 may be integrated with the second stator holder 423B. The first stator holder 423A and the second stator holder 423B may hold the stator 468 in place in the motor housing 423. The first rotor 464 may be positioned in the first stator holder 423A. The second rotor 466 may be positioned in the second stator holder 423B.

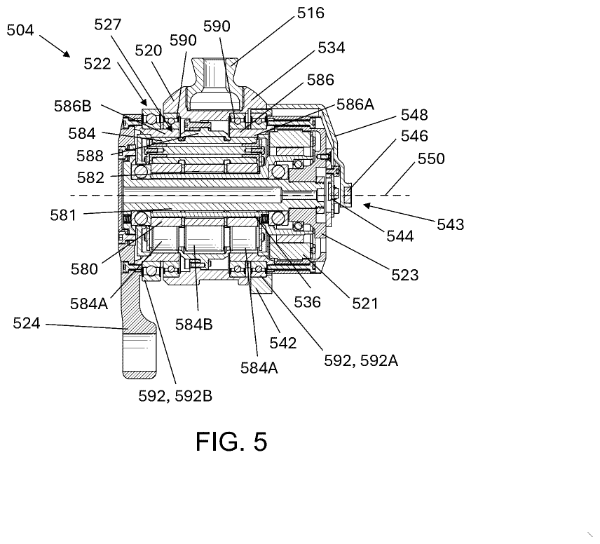

FIG. 5 illustrates a cross-sectional view of a joint mechanism 504 for a prosthetic device, such as prosthetic devices 100, 400. The joint mechanism 504 can include any of the features of the joint mechanisms 104, 404, and the joint mechanisms 104, 404 can include any of the features of joint mechanism 504. The joint mechanism 504 may include a drive unit 522. The drive unit 522 may include drive transmission 527. The drive transmission 527 may include a compound-planetary drive transmission. The drive transmission 527 may include a traction or friction drive transmission.

The drive unit 522 can include an input shaft 536. The input shaft 536 may be coupled to a motor 521 and the drive transmission 527. The motor 521 may include a radial flux motor or an axial flux motor, or an electric motor, such as a brushless direct current (DC) motor. The motor 521 may be coupled to the motor housing 523 of the housing 520. The input shaft 536 may extend axially along a transverse axis 550.

The drive transmission 527 may include a first sun roller 580, a second sun roller 582, planet rollers 584, an outer ring annulus 586, and/or an inner ring annulus 588. The motor 521 may rotate the input shaft 536 about the transverse axis 550. The input shaft 536 may be coupled to the first sun roller 580 or the input shaft 536 and the first sun roller 580 may be integrated together. The first sun roller 580 may be positioned so the first sun roller 580 contacts outer portions 584A of the planet rollers 584. Accordingly, when the first sun roller 580 rotates about the transverse axis 550, the first sun roller 580 may cause the planet rollers 584 to rotate.

The planet rollers 584 may be hollow. The planet rollers 584 may be positioned so the outer portions 584A contact the outer ring annulus 586. The outer ring annulus 586 may be coupled to the motor housing 523 and the output arm 524. The planet rollers 584 may be positioned so an inner portion 584B of each planet roller 584 contacts the inner ring annulus 588. The inner ring annulus 588 may be coupled to the drive unit housing 534 of the housing 520. The drive unit housing 534 may be coupled to the proximal connector 516. Accordingly, when the first sun roller 580 causes the planet rollers 584 to rotate, the planet rollers 584 may cause the inner ring annulus 588 to rotate about the transverse axis 550 relative to the outer ring annulus 586. Therefore, the drive unit housing 534 and the proximal connector 516 may rotate relative to the motor housing 523 and the output arm 524. The motor housing 523 and the output arm 524 may be coupled to a shank portion of a prosthetic device, and the proximal connector 516 may be connected to another adjacent prosthetic portion, such as a common socket, (not shown) that is mountable to a limb segment (e.g., leg stump) of a user. Accordingly, rotation of the drive unit housing 534 and the proximal connector 516 relative to the motor housing 523 and the output arm 524 may cause the shank portion of the prosthetic device to rotate relative to the limb segment of the user.

The second sun roller 582 may be positioned concentrically around the input shaft 536. The second sun roller 582 may not contact the input shaft 536. Accordingly, the second sun roller 582 may rotate freely about the transverse axis 550 and/or the input shaft 536. The second sun roller 582 may contact the inner portion 584B of each planet roller 584. Accordingly, when the planet rollers 584 rotate, the planet rollers 584 may cause the second sun roller 582 to rotate about the transverse axis 550 and/or the input shaft 536. The second sun roller 582 may maintain uniform pressure against the inner portion 584B of each planet roller 584 so the inner portion 584B of each planet roller 584 maintains sufficient contact with the inner ring annulus 588 to maintain the required friction or traction between the inner portion 584B of each planet roller 584 and the inner ring annulus 588 so the inner portion 584B of each planet roller 584 may rotate the inner ring annulus 588 without slippage (e.g., backlash).

The drive transmission 527 may include a central shaft (e.g., central yoke) 581. The central shaft 581 may extend through the input shaft 536. The central shaft 581 may functionally and/or structurally connect each portion 586A, 586B of the outer ring annulus 586 together so the portions 586A, 586B rotate together. The first portion 586A of the outer ring annulus 586 may be coupled to the motor housing 523 and the second portion 586B may be coupled to the output arm 524. The central shaft 581 may couple the motor housing 523 and the output arm 524 together in order to functionally and/or structurally connect each portion 586A, 586B of the outer ring annulus 586 together.

A diameter of the outer portions 584A of the planet rollers 584 may be different than a diameter of the inner portion 584B of the planet rollers 584. The diameter of the outer portions 584A of the planet rollers 584 may be smaller than the diameter of the inner portion 584B of the planet rollers 584. Accordingly, the diameter of the inner portion 584B of the planet rollers 584 may be larger than the diameter of the outer portions 584A of the planet rollers 584. The diameter of the inner portion 584B of the planet rollers 584 may be modified to modify a gear ratio (e.g., reduction ratio) of the drive unit 522.

The joint mechanism 504 may include inner bearings 590. The inner bearings 590 may be positioned between the outer ring annulus 586 and the drive unit housing 534. The inner bearings 590 may allow the drive unit housing 534 to rotate relative to the outer ring annulus 586.

The motor housing 523 and/or the first portion 586A of the outer ring annulus 586 may be coupled to the shank structure via a support block 542. The support block 542 may include a pillow block. The output arm 524 may be coupled to the shank structure via a rigid connection or a compliant connection (e.g., transmission). If the output arm 524 is coupled to the shank structure via a compliant connection (e.g., transmission), the motor housing 523, the output arm 524, the outer ring annulus 586, and/or any other components fixedly coupled to aforementioned components must be able to rotate relative to the shank structure. Accordingly, in some embodiments, the joint mechanism 504 may include outer bearings 592 positioned between the outer ring annulus 586 and the shank structure. A first outer bearing 592A may be positioned between the motor housing 523 and the support block 542 and/or the first portion 586A of the outer ring annulus 586 and the support block 542. A second outer bearing 592B may be positioned between the portion 586B of the outer ring annulus 586 and the shank structure.

The drive unit 522 may include a sensor assembly 543. The sensor assembly 543 may include a sensor 544. The sensor 544 may measure an angle between the proximal connector 516 (or the limb segment of the user) and the shank portion of the prosthetic device. The sensor 544 may be coupled to the outside of the motor housing 423. The sensor 544 may include a rotary magnetic sensor, for example, a hall-effect type rotary sensor. The drive unit 522 may include a magnet 546. The magnet 546 may be positioned outside of the motor housing 123 so the magnet 546 is coaxially aligned with the transverse axis 550. The drive unit 522 may include a bracket 548. The bracket 548 may hold the magnet 546 is position. The bracket 548 may be coupled to the drive unit housing 534 and/or the proximal connector 516. Accordingly, when the drive unit housing 534 and/or the proximal connector 516 rotate relative to the motor housing 523 and/or the shank portion, the magnet 546 may rotate relative to the sensor 544. When the magnet 546 rotates, the sensor 544 may detect changes in a magnetic field generated by the magnet 546 in order to measure the angle between the proximal connector 516 (or the limb segment of the user) and the shank portion of the prosthetic device. If the output arm 524 is coupled to the shank segment via a compliant connection (e.g., transmission), the drive unit 522 may include a second sensor (not shown) in order to accurately measure the angle between the proximal connector 516 (or the limb segment of the user) and the shank portion of the prosthetic device.

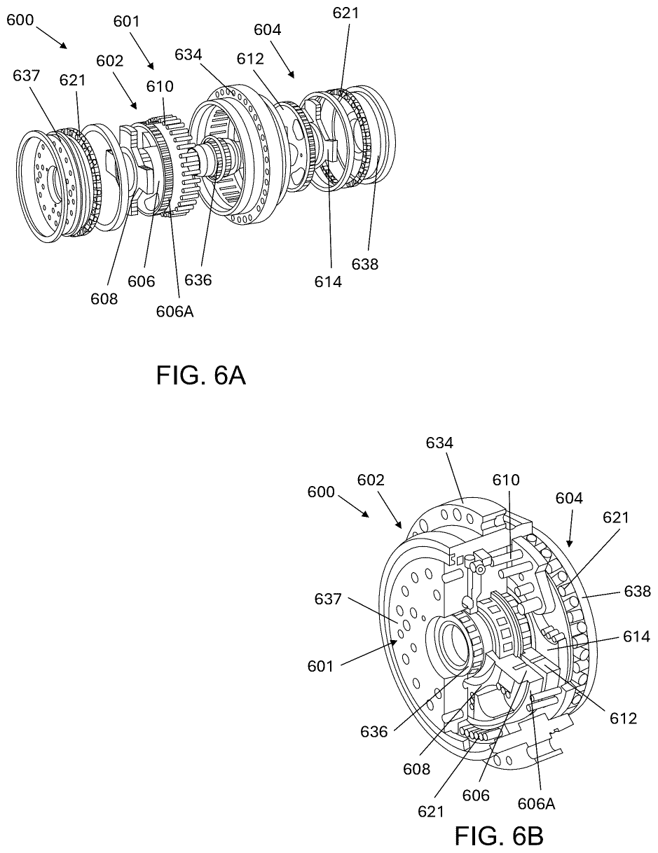

FIGS. 6A and 6B illustrate a drive unit 600 of a joint mechanism for a prosthetic device, such as prosthetic devices 100, 400. FIG. 6A illustrates an exploded view of the drive unit 600. FIG. 6B illustrates a partial cross-sectional assembled view of the drive unit 600. The drive unit 600 can include any of the features of the drive units 122, 422, 522, and the drive units 122, 422, 522 can include any of the features of the drive unit 600.

The drive unit 600 may include a drive transmission 601,, an input shaft 636, and/or a first output 637. The drive unit 600 may be positioned in (e.g., rigidly coupled to) a drive unit housing 634. The drive transmission 601 may be a cycloid drive transmission. The drive transmission 601 may couple the input shaft 636 to the first output 637. The drive transmission 601 may transfer rotation of the input shaft 636 to the first output 637. The drive transmission 601 may include a first stage 602. The first stage 602 may include a gear 606 and a transformation member 608. The gear 606 may be a trochoidal gear such that teeth 606A of the gear 606 form a trochoidal shape.

The gear 606 may be eccentrically coupled to the input shaft 636. Accordingly, when the input shaft 636 rotates, the gear 606 may rotate and translate in a cycloidal path about the input shaft 636 in an opposite direction as the input shaft 636. As the gear 606 rotates, the teeth 606A may interact with pins 610 positioned circumferentially around the gear 606 and coupled to the drive unit housing 634. The gear 606 may rotate a portion of a revolution each time the input shaft 636 rotates an entire revolution. The interaction between the pins 610 and the teeth 606A of the gear 606 may generate torque.

The gear 606 may be coupled to the transformation member 608, and the transformation member 608 may be coupled to the first output 637. The first output 637 may be a shaft or a flange. The transformation member 608 may transform (e.g., convert) the eccentric and/or cycloidal rotation of the gear 606 to non-eccentric rotation (e.g., rotation about a same axis as the input shaft 636) such that the first output 637 rotates about the same axis as the input shaft 636. The transformation member 608 may transfer the torque generated by the interaction between the teeth 606A of the gear 606 and the pins 610 to the first output 637.

The drive transmission 601 may include a reduction (e.g., gear) ratio. The reduction ratio may be calculated as a ratio of the number of teeth 606A to the number of pins 610 minus the number of teeth 606A. For example, a first stage 602 with a gear 606 with 11 teeth 606A and 12 pins 620 may have a reduction ratio of 12:1. The reduction ratio of the first stage 602 may include a ratio of 21:1, 23:1, 25:1, 27:1, 29:1, 30:1, 31:1, 33:1, 35:1, 37:1, 39:1, 41:1, 43:1, 45:1, 47:1, 49:1, 50:1, 51:1, 53:1, 55:1, 59:1, 60:1, 61:1, 63:1, 65:1, 67:1, 69:1, and/or any value between the aforementioned values. In some embodiments, the reduction ratio may be a ratio between 30:1 and 50:1. In some embodiments, the reduction ratio may be a ratio between 35:1 and 47:1. In some embodiments, the reduction ratio may be a ratio between 25:1 and 60:1.

The drive unit 600 may include a second stage 604. The second stage 604 may be coupled to a second output 638 on an opposite side of the drive unit housing 634. The second stage 604 may include any of the features and/or components of the first stage 602. The second stage 604 may include a gear 612 and a transformation member 614. The gear 612 of the second stage 604 may be coupled to the input shaft 636 such that the gear 612 is offset from the gear 606 by 180 degrees. Accordingly, as the gear 612 and the gear 606 rotate and translate in a cycloidal path about the input shaft 636, the translation of the gear 612 may be an opposite direction of the translation of the gear 606. Therefore, the gear 612 of the second stage 604 may offset the translation of the gear 606 of the first stage 602 to reduce or minimize vibration of the drive unit 600.

The drive unit 600 may include bearings 621. The bearings 621 may rotatably couple the first output 637 and/or the second output 638 to the drive unit housing 634.

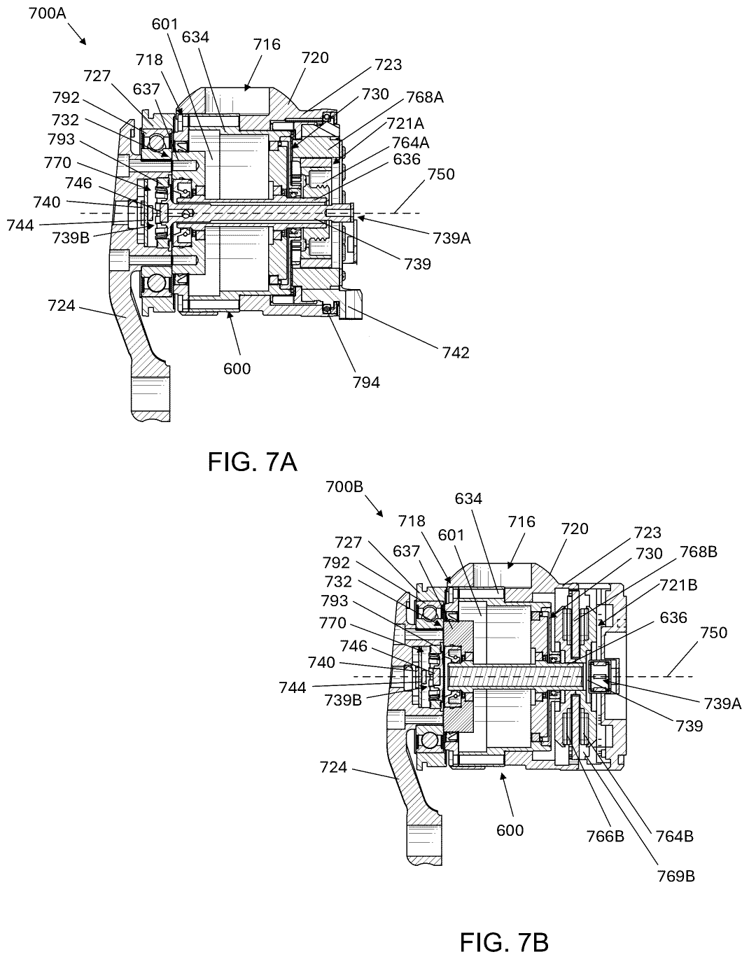

FIGS. 7A and 7B illustrate cross-sectional views of joint mechanisms 700A, 700B with the drive unit 600 for a prosthetic device, such as prosthetic devices 100, 400. FIG. 7A illustrates a cross-sectional view of the joint mechanism 700A having a radial flux motor 721A. FIG. 7B illustrates a cross-sectional view of the joint mechanism 700B having an axial flux motor 721B. The joint mechanisms 700A, 700B may include any of the features of the joint mechanisms 104, 404, 504, and the joint mechanisms 104, 404, 504 may include any of the features of the joint mechanisms 700A, 700B.

As shown in FIGS. 7A and 7B, the joint mechanisms 700A, 700B may include an actuation assembly 718A, a housing 720, the drive unit 600, and/or an output arm 724. For clarity, the drive transmission 601 of the drive unit 600 is shown generically.

The housing 720 may include the drive unit housing 634, a motor housing 723, and an output housing 727. The motor housing 723 may include a recess 716 that may couple with a proximal connector, such as proximal connector 116. The motors 721A, 721B may be positioned in the motor housing 723 of the housing 720. The drive unit housing 634 may be rigidly coupled to the motor housing 723 such that the motors 721A, 721B are coupled to the drive unit 600 on a first side 730 of the drive unit 600. The output housing 727 may be rigidly coupled to the motor housing 723 and/or the drive unit housing 634 on a second side 732 of the drive unit 600.

The motors 721A, 721B may be coupled to the input shaft 636 of the drive unit 600. The motors 721A, 721B may rotate the input shaft 636 about a transverse axis 750 extending through the drive unit 600. As discussed above with reference to FIGS. 6A and 6B, the drive transmission 601 may transfer rotation of the input shaft 636 to the first output 637. The first output 637 of the drive unit 600 may be coupled to the output arm 724. The output arm 724 may be rotatably coupled to the output housing 727 with a first bearing 792. When the first output 637 rotates about the transverse axis 750, the first output 637 may cause the output arm 724 to rotate relative to the drive unit housing 634 and the motor housing 723 about the transverse axis 750. Accordingly, the output arm 724 may rotate a shank structure, such as shank structure 110, relative the drive unit housing 634 and the motor housing 723 when the first output 637 rotates the output arm 724. The output arm 724 may include a rigid output arm or a compliant connection element (e.g., transmission). The reduction ratio of the drive transmission 601 may be selected to reduce or minimize a weight and/or volume of the drive transmission 601, a weight and/or volume of the motors 721A, 721B required to operate the drive unit 600 during operation of the prosthetic device, and/or a speed of the motors 721A, 721B required to rotate the output arm 724 during operation of the prosthetic device.

The input shaft 636 may be hollow. The joint mechanisms 700A, 700B may include a measurement shaft 739 extending axially through the input shaft 636 such that a second end 739B of the measurement shaft 739 opposite a first end 739A is positioned in a recess 770 in the output arm 724. The measurement shaft 739 may be coupled to the motor housing 723. For example, the first end 739A may be coupled to the motor housing 723. Accordingly, the measurement shaft 739 may not rotate relative to the housing 720 when the output arm 724 rotates relative to the housing 720. The second end 739B of the measurement shaft 739 may be rotatably coupled to the output arm 724 via a second bearing 793.

A controller 740 may be positioned in the recess 770 and coupled to the output arm 724. The controller 740 may be coupled to an encoder (e.g., sensor) 744. The encoder 744 may include a magnetic encoder, a mechanical encoder, and/or an optical encoder. The encoder 744 may include an incremental encoder or an absolute encoder. The encoder 744 may include a single turn magnetic encoder. When the output arm 724 rotates about the transverse axis 750, the encoder 744 may rotate about the transverse axis 150 with the output arm 724. The encoder 744 may measure a rotational position of the encoder 744 relative to the measurement shaft 739 about the transverse axis 750 to determine a rotational position of the output arm 724 and/or the first output 637 of the drive unit 600 relative to the motors 721A, 721B (and therefore the housing 720). The rotational position of the output arm 724 and/or the first output 637 relative to the motors 721A, 721B may be used to determine an angle of flexion of the prosthetic device. The controller 740 may control the motors 721A, 721B based at least in part on the angle of flexion of the prosthetic device. The encoder 744 may include a high-speed encoder (e.g., sensor) and/or a low-speed encoder. Accordingly, the encoder 744 may enable full field-oriented control of the motor commutation of the motors 721A, 721B by the controller 740.

The measurement shaft 739 may include a magnet 746 coupled to the second end 739B of the measurement shaft 739. When the encoder 744 rotates relative to the measurement shaft 739 and the magnet 746, the encoder 744 may detect changes in a magnetic field generated by the magnet 746 in order to measure the angle of flexion of the prosthetic device. The controller 740 and/or the encoder 744 may be formed on and/or coupled to a printed circuit board (PCB).

As shown in FIG. 7A, the housing 720 of the joint mechanism 700A may be coupled to the shank structure of the prosthetic device with a support block 742. The support block 742 may be rotatably coupled the housing 720 via a third bearing 794.

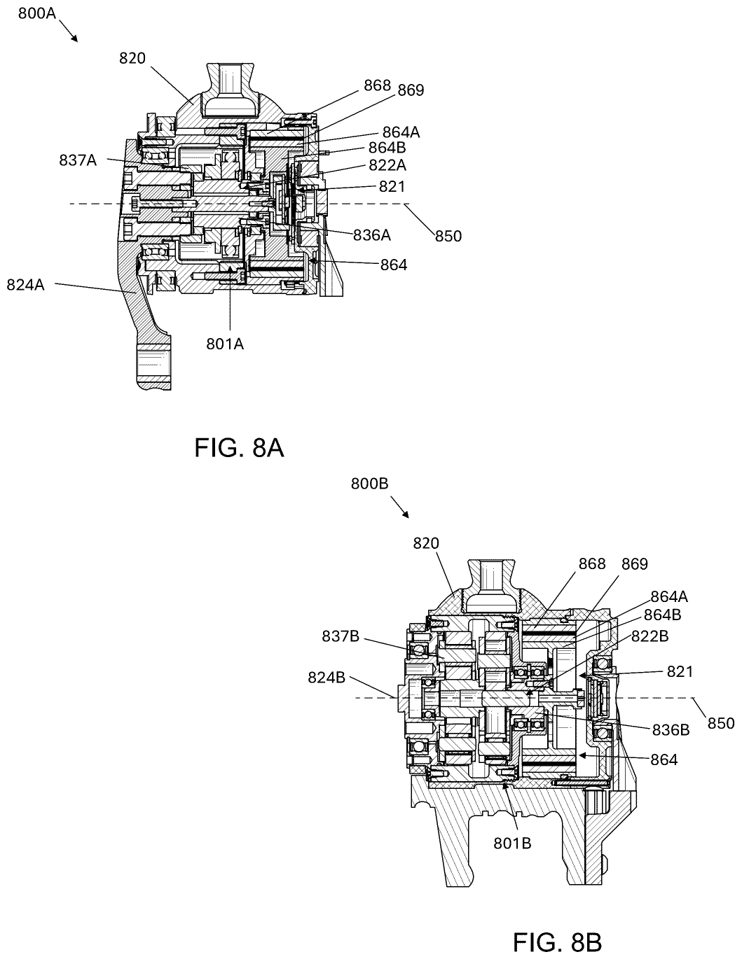

As discussed above, the motor 721A of the joint mechanism 700A may be a radial flux motor. In some embodiments, the motor 721A may be a motor with PCB windings, such as the motor 821 (see FIGS. 8A and 8B). The motor 721A of the joint mechanism 700A may include a rotor 764A and/or a stator 768A. The rotor 764A may be coupled to the input shaft 636 of the drive unit 600. The rotor 764A may extend circumferentially around the input shaft 636. The stator 768A may be positioned circumferentially around the rotor 764A. The stator 768A may be coupled to the motor housing 723. The rotor 764A may include magnets mounted to a carrier. Current may be applied to the stator 768A to generate a magnetic field. The magnetic field may cause rotation of the rotor 764A and the input shaft 636.

As discussed above, the motor 721B of the joint mechanism 700B may be an axial flux motor. The motor 721B may include any of the features of the motor 421, and the motor 421 may include any of the features of the motor 721B.

The motor 721B may include a first rotor 764B, a second rotor 766B, and/or a stator 768B. The first rotor 764B and the second rotor 766B may be coupled together. The stator 768B may be positioned between the first rotor 764B and the second rotor 766B. The first rotor 764B and the second rotor 766B may be positioned on opposite sides of the stator 768B. The first rotor 764B and the second rotor 766B may be coupled to the input shaft 636 such that when the stator 768B rotates the first rotor 764B and the second rotor 766B, the first rotor 764B and the 766B may rotate the shaft 636.

The stator 768B may include a printed circuit board (PCB). Making the stator 768B a PCB may increase the manufacturing repeatability of the stator 768B and/or the axial flux motor 721B, reduce or minimized a cost of manufacturing of the stator 468 and/or the axial flux motor 721B, and/or allow the axial flux motor 721B to have a high torque density. Additionally, making the stator 768B a PCB may reduce or minimize a weight of the stator 768B.

The first rotor 764B, the second rotor 766B, and/or the stator 768B may be substantially flat. The first rotor 764B, the second rotor 766B, and/or the stator 768B may be positioned perpendicular to the transverse axis 750.

The stator 768B may be coupled the motor housing 723 via a collar 769B such that the stator 768B does not rotate relative to the motor housing 723. The collar 769B may be a heat sink. The collar 769B may transfer heat generated by the stator 768B to the motor housing 723 to dissipate heat from the stator 768B.

FIGS. 8A and 8B illustrate cross-sectional views of joint mechanisms 800A, 800B with a motor 821 having windings 869 formed from PCB, for a prosthetic device, such as prosthetic devices 100, 400. FIG. 8A illustrates a cross-sectional view of the joint mechanism 800A with the motor 821 and an example of a first drive transmission 822A. FIG. 8B illustrates a cross-sectional view of the joint mechanism 800B with the motor 821 and an example of a second drive transmission 822B. The joint mechanism 800 may include any of the features of the joint mechanisms 104, 404, 504, 700A, 700B, and the joint mechanisms 104, 404, 504, 700A, 700B may include any of the features of the joint mechanism 800.

With reference to FIGS. 8A and 8B, the motor 821 may be coupled to the input shafts 836A, 836B of the drive units 801A, 801B. The motor 821 may rotate the input shafts 836A, 836B about a transverse axis 850 extending through the joint mechanisms 800A, 800B.

As shown in FIG. 8A, the drive unit 801A may include the first drive transmission 822A. The first drive transmission 822A may include a cycloid drive transmission, such as drive transmission 601, or a strain wave drive transmission. The first drive transmission 822A may transfer rotation of the input shaft 836A to an output 837A of the drive unit 801A. The output 837A may be coupled to an output arm 824A. When the output 837A rotates about the transverse axis 850, the output 837A may cause the output arm 824A to rotate relative to a housing 820 of the joint mechanism 800A. Accordingly, the output arm 824A may rotate a shank structure, such as shank structure 110, relative to the housing 820 when the output 837A rotates the output arm 824A. The output arm 824A may include a rigid output arm or a compliant transmission element.

As shown in FIG. 8B, the drive unit 801B may include the second drive transmission 822B. The second drive transmission 822B may include a traction or friction drive transmission, such as drive transmissions 200, 427, 527. The second drive transmission 822B may include one (1) stage, two (2) stages, three (3) stages, four (4) stages, five (5) stage, and/or six (6) stages. The second drive transmission 822B may include any of the features of the drive transmissions 200, 427, 527, and the drive transmissions 200, 427, 527 may include any of the features of the second drive transmission 822B. The second drive transmission 822B may transfer rotation of the input shaft 836B to an output 837B of the drive unit 801B. The output 837B may be coupled to an output arm (not shown in FIG. 8B), such as output arm 824A. When the output 837B rotates about the transverse axis 850, the output 837B may cause the output arm to rotate relative to the housing 820 of the joint mechanism 800B. Accordingly, the output arm may rotate a shank structure, such as shank structure 110, relative to the housing 820 when the output 837B rotates the output arm 824B. The output arm may include a rigid output arm or a compliant transmission element.

The motor 821 may include a rotor 864 and a stator 868. The rotor 864 may be coupled to the input shafts 836A, 836B of the drive units 801A, 801B. The rotor 864 may extend circumferentially around the input shafts 836A, 836B. The stator 868 may be positioned circumferentially around the rotor 864. The stator 868 may be coupled to a motor housing 823 of the housing 820. The motor 821 may include windings 869 positioned between the rotor 864 and the stator 868. The windings 869 may include a printed circuit board (PCB) or printed circuit laminate. The PCB or printed circuit laminate may be flexible such that the PCB or printed circuit laminate may be rolled into a cylinder comprising one or more layers of the PCB or printed circuit laminate to form the windings 869.

The rotor 864 may include a magnet 864A mounted to a carrier 864B. The magnet 864A may include plurality of permanent magnet segments, or a selectively magnetized piece or metal, such as a rare earth metal. The carrier 864B may be coupled to the input shafts 836A, 836B. Current may be applied to the stator 868 to generate a magnetic field. The magnetic field may cause rotation of the rotor 864 and the input shafts 836A, 836B.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. An actuation assembly for a joint mechanism of a prosthetic device, the actuation assembly comprising:
an output arm;
a motor; and
a drive unit comprising an input coupled to the motor, an output coupled to the output arm, and a drive transmission, wherein the drive transmission is configured to transmit rotation of the input to the output,
wherein the motor and the drive unit are axially aligned, and the drive transmission comprises a traction transmission or a friction transmission.

2. The actuation assembly of claim 1, wherein a reduction ratio of the drive transmission is between 35:1 and 80:1.

3. The actuation assembly of claim 1, wherein the motor comprises a brushless motor.

4. The actuation assembly of claim 1, wherein the motor comprises an axial flux motor.

5. The actuation assembly of claim 1, wherein the motor comprises a radial flux motor.

6. The actuation assembly of claim 1, wherein windings of the motor comprise flexible printed circuit board.

7. The actuation assembly of claim 1, wherein the prosthetic device comprises a lower limb prosthetic device.

8. The actuation assembly of claim 1, wherein a reduction ratio of the drive transmission is between 25:1 and 60:1.

9. The actuation assembly of claim 1, wherein the output arm comprises a compliant transmission.

10. The actuation assembly of claim 9, wherein the compliant transmission comprises two links, wherein a first link of the two links is compliant.

11. The actuation assembly of claim 10, wherein the compliant transmission comprises a single flexible output arm.

12. The actuation assembly of claim 1, wherein the output arm is rigid.

13. A joint mechanism for a prosthetic device, the joint mechanism comprising:
a housing comprising a prosthetic connector; and
an actuation assembly positioned in the housing, the actuation assembly comprising:
an output arm;
a motor; and
a drive unit comprising an input coupled to the motor, an output coupled to the output arm, and a drive transmission, wherein the drive transmission is configured to transmit rotation of the input to the output,
wherein a rotational axis of the motor and a rotation axis of the drive unit are parallel, and wherein the drive transmission comprises a traction transmission or a friction transmission.

14. The joint mechanism of claim 13, wherein the drive unit comprises a measurement shaft, and an encoder configured to measure a rotation position of the measurement shaft.

15. The joint mechanism of claim 13, wherein the housing comprises a drive unit housing coupled to a motor housing, wherein the drive unit is positioned in the drive unit housing and the motor is positioned in the motor housing.

16. The joint mechanism of claim 15, wherein the prosthetic connector is coupled to the motor housing.

17. The joint mechanism of claim 13, wherein the prosthetic device comprises a lower limb prosthetic device.

18. The joint mechanism of claim 13, wherein the drive transmission comprises a planetary drive transmission.

19. The joint mechanism of claim 18, wherein the planetary drive transmission comprises a plurality of stages.

20. The joint mechanism of claim 13, wherein the drive transmission comprises a compound-planetary drive transmission.
